(19) United States
(12) Patent Application Publication
(10) Pub. No.: US 2018/0021456 A1
(43) Pub. Date: Jan. 25, 2018

(54) SELF-ASSEMBLED TARGETED INCLUSION COMPLEXES FOR DRUG DELIVERY

(71) Applicant: UNIVERSITY OF SOUTH FLORIDA, Tampa, FL (US)

(72) Inventors: SHU-FENG ZHOU, TAMPA, FL (US); JUN TAN, TAMPA, FL (US); JUAN-JUAN YIN, TAMPA, FL (US)

(21) Appl. No.: 15/547,815

(22) PCT Filed: Feb. 4, 2016
(86) PCT No.: PCT/US2016/016513

§ 371 Date: Aug. 1, 2017

Related U.S. Application Data
(60) Provisional application No. 62/111,803, filed on Feb. 4, 2015.

(51) Int. Cl. A61K 47/69; A61K 31/704; C08B 37/16

(52) U.S. Cl. CPC A61K 47/6951; C08B 37/0015; A61K 31/704

(57) ABSTRACT

Inclusion complexes are provided containing a targeted carrier non-covalently associated with an active agent. The targeted carrier includes an inclusion host that binds the active agent or a binding partner attached to the active agent. The targeted carrier includes a targeting moiety attached to the inclusion host or to a polymer or linker attached thereto. Pharmaceutical formulations of the inclusion complexes are provided. Methods of making the inclusion complexes and formulations thereof are provided. Methods of using the inclusion complexes and formulations are provided. In some embodiments the inclusion complex includes a progesterone or estrogen targeting moiety that targets the inclusion complex to cancer cells. The targeting can cause internalization of the active agent in the target cells. In some embodiments the active agent is an anthracycline such as doxorubicin and the target cells are cancer cells.

### BACKGROUND

Cancer remains one of the most devastating diseases worldwide. According to the cancer report by American Cancer society, cancer remains the second leading cause of death in US accounting for approximately 5.8 million of deaths in 2013 (Siegel, Naishadham et al. 2013). Current management of cancer, including surgery, chemotherapy and radiation, faces multiple challenges. These challenges include severe adverse side effect and poor effectiveness, especially when metastasis occurs. Targeted therapies that can deliver drugs selectively to the cancer cells without systemic toxicity are well known to improve survival rate and the quality of life for cancer patients.

Cancer therapies have advanced considerably during the last few decades. However, they are also still hampered by nonspecific delivery of anti-tumor agents to normal cells, resulting in horrendous side effects for patients. This lack of specificity also results in lower efficacy of treatments due to the want of a capability to deliver active agents in a focused manner where they are most needed, e.g. to cancer cells alone.

Therefore, it would be desirable to develop new carriers, formulations, and methods for targeted delivery and internalization of active agents to cancer cells that minimize off-target toxicity.

### SUMMARY

Inclusion complexes are provided containing a targeted carrier non-covalently associated with an active agent or precursor thereof. The targeted carrier can contain an inclusion host having a targeting moiety conjugated thereto. The targeting moiety can be conjugated to a linker or polymer conjugated to the inclusion host. In various embodiments the inclusion host is cyclodextrin; the targeting moiety is estrogen or progesterone; and the precursor is the doxorubicin active agent conjugated to an adamantane binding partner.

The active agent or precursor thereof can non-covalently associate with the inclusion host via π-bonding, cation-π, Van der Walls, or hydrogen-bonding interactions. The precursor can contain a conjugate of the active agent and a binding partner that non-covalently binds to the inclusion host, for example via -bonding, cation-π, Van der Walls, or hydrogen-bonding interactions.

A variety of inclusion hosts can be useful for the inclusion complexes described herein. The inclusion host can be a crown ether, cyclodextrin, calixarene, or cucurbituril. The cyclodextrin can be α-cyclodextrin, β-cyclodextrin, or γ-cyclodextrin. The inclusion complex can bind to one or more defined binding partners, e.g. a binding partner that can bind to the inclusion host with high specificity. The binding partner can be a phenyl group, naphthol derivative, lipid, or a cage-like cycloalkane. The binding partner can be adamantane, diamantine, or iceane.

The inclusion complex can be used to deliver a variety of active agents to specific cells, e.g. by targeting a specific cell surface receptor that results in internalization of the active agent. The active agent can be a therapeutic, prophylactic, or diagnostic agent. The targeting moiety can be a protein, a peptide, a nucleic acid, or a small molecule. The targeting moiety can target a cell surface receptor that results in internalization of the active agent. The targeting moiety can be estrogen or progesterone.

The targeted carrier can contain a polymer covalently associated with the inclusion host, and in some embodiments the targeting moiety can be covalently associated with the polymer. The polymer can be a biodegradable polymer such as a poly(alkanoic acid) or a copolymer or derivative thereof. The polymer can be a poly(alkylene oxide) such as poly(ethylene oxide), poly(propylene oxide), or a copolymer or derivative thereof.

Pharmaceutical formulations are provided containing an effective amount of the inclusion complexes. The formulations can include one or more excipients such as sugars or sugar alcohols, buffering agents, preservatives, solvents, antioxidants, chelating agents, water-soluble natural or synthetic polymers, cryoprotectants, lyoprotectants, surfactants, bulking agents, or stabilizing agents. In some embodiments the formulations are for treating cancer. Methods of treating or preventing a disease or disorder, including cancer, by administering the formulations are also provided.

Methods of making the inclusion complexes are provided. The methods can include self-assembly or a targeted carrier having an inclusion host and an active agent. The active agent or a precursor thereof can non-covalently associate with the inclusion host. The method can include preparing the by providing an inclusion host having a reactive coupling group, such as an amine, covalently conjugated thereto and reacting the targeting moiety to form a covalent bond with the reactive coupling group to form the targeted carrier.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 depicts one embodiment of an inclusion complex containing a doxorubicin active agent conjugated to an adamantane binding partner. The adamantane binds non-covalently to the targeted carrier containing a β-cyclodextrin conjugated to an estrogen targeting moiety.

FIG. 2 is a schematic of one method of making the inclusion complex depicted in FIG. 1.

FIG. 3 is a ¹H NMR spectrum (800 MHz, d₄-DMSO, T=298 K) of the inclusion complex CDE1-Ada-DOX containing an estrogen targeted cyclodextrin and an adamantane-doxorubicin conjugate.

FIG. 4 is a HR-MALDI-TOF spectrum of the inclusion complex CDE1-Ada-DOX containing an estrogen targeted cyclodextrin and an adamantane-doxorubicin conjugate.

FIG. 5 is a fluorescence emission spectra of the inclusion complex CDE1-Ada-DOX containing an estrogen targeted cyclodextrin and an adamantane-doxorubicin conjugate. (cAda-DOX=50 μM) at different CDE₁ concentrations of 0.17, 0.26, 0.35, 0.44, 0.53, 0.62, 0.71, 0.79, and 0.88 mM, with increasing step of 0.08 mM. Samples in fluorescence measurements are excited at λex=485 nm.

FIG. 6 is a circular dichroism spectrum of the inclusion complex CDE1-Ada-DOX containing an estrogen targeted cyclodextrin and an adamantane-doxorubicin conjugate.

FIG. 7 is a bar graph of the cumulative drug release profiles of Ada-Dox and CDE1-Ada-Dox. The release of Ada-Dox was determined by a dialysis method. The released Ada-Dox was quantified by microplate reader at λEx=490 nm and λEm=600 nm. A calibration curve was prepared using different concentrations of free Ada-Dox.

FIG. 8 is a bar graph of the absolute drug release of Ada-Dox and CDE1-Ada-Dox. The release of Ada-Dox was determined by a dialysis method. The released Ada-Dox was quantified by microplate reader at λEx=490 nm and λEm=600 nm. A calibration curve was prepared using different concentrations of free Ada-Dox.

FIG. 9 is a graph of the fluorescence intensity of various samples of CDE₁ and CDE₁-Ada-DOX with host/guest molar concentration ratio of 1:1, 3:1 and 5:1 at 1 μM of Ada-DOX in MCF-7 cells from flow cytometric analysis.

FIG. 10 is a bar graph of the fluorescence intensity of Ada-Dox for MCF-7 cells incubated with 1 targeted (CDE1) and non-targeted (NCDE1) inclusion complexes (1 μm) with a 1:1 ratio of adamantane-doxorubicin (Ada-DOX) at 2, 4, and 6 hours. Cells incubated with DMEM medium only were used as the control.

FIG. 11 is a bar graph of the fluorescence intensity of Ada-Dox for MCF-7 cells incubated with targeted (CDE₁) and non-targeted (NCDE₁) inclusion complexes (1 μm) with a 1:2 ratio of adamantane-doxorubicin (Ada-DOX) at 2, 4, and 6 hours. Cells incubated with DMEM medium only were used as the control.

FIG. 12 is a bar graph of the fluorescence intensity of Ada-Dox for MCF-7 cells incubated for four hours with (from left to right) DMEM medium, 5 μm non-targeted NCDE₁-Ada-DOX at a ratio of 1:1, 5 μm of targeted CDE₁-Ada-DOX at a ratio of 1:1, 5 μm of non-targeted NCDE₁-Ada-DOX at a ratio of 1:2, and 5 μm of targeted CDE₁-Ada-DOX at a ratio of 1:2.

FIG. 13 is a graph depicting the change in drug uptake (% change) of targeted CDE₁-Ada-DOX relative to non-targeted NCDE₁-Ada-DOX as a function of the concentration, ratio of targeted carrier to drug, and incubation time. The arrows indicate downward (decreased uptake relative to NCDE₁-Ada-DOX) or upward (increased uptake relative to NCDE₁-Ada-DOX).

FIGS. 14A-14C are TEM/SEM characterizations of CDE₁-Ada-DOX and CDE₁. A solution of CDE₁ or CDE₁-Ada-DOX at 1.8 mM in water and DMF (v/v 1:1) dried in a vacuum oven at 35° C. overnight. FIG. 14A is a TEM image of CDE1-Ada-DOX. FIG. 14B is a TEM image of CDE₁ and its image in high magnification in the corner. FIG. 14C is an SEM image of CDE1.

FIG. 15 depicts the structure of CDE₁-Ada-DOX as determined from the TEM image.

FIG. 16 depicts the structure of free CDE₁ confirmed by TEM and SEM.

FIG. 17 is a bar graph of the fluorescence intensity for competition assay of CDE₁-Ada-DOX at 2 μM in MCF-7 cells in the presence of estrone and tamoxifen at 0.1-10 μM. The results are represented as means±SD from triplicate determinations.

FIG. 18 is a graph of the cholesterol depletion from the cell membrane of human breast cancer cells MCF-7 cells after the treatment with saturated CDE1 solution; Natural ligand E₁ (40 μM); Positive control methyl-β-cyclodextrin (Methyl-CD) and β-cyclodextrin (beta-CD); Doxorubicin (DOX) at 0.5, 2.5 μM; Adamantane (AD) at 10, 100 μM; and the drug complex CDE1-Ada-DOX in different host/guest ratio of 1:1, 1:3 and 1:5 respectively, at the host concentration of 10 μM. Cell cultures were treated for 2 h at 37° C. to deplete cholesterol. Cells were then washed twice in serum-free media and lysed. Cholesterol levels were detected using EnzyChrom™ cholesterol assay kit.

FIG. 19 is a graph of the cholesterol depletion from the cell membrane of human normal breast cells MCF-10A cells after the treatment with saturated CDE1 solution; Natural ligand E₁ (40 μM); Positive control methyl-β-cyclodextrin (Methyl-CD) and β-cyclodextrin (beta-CD); Doxorubicin (DOX) at 0.5, 2.5 μM; Adamantane (AD) at 10, 100 μM; and the drug complex CDE1-Ada-DOX in different host/guest ratio of 1:1, 1:3 and 1:5 respectively, at the host concentration of 10 μM. Cell cultures were treated for 2 h at 37° C. to deplete cholesterol. Cells were then washed twice in serum-free media and lysed. Cholesterol levels were detected using EnzyChrom™ cholesterol assay kit.

FIG. 20 is a graph of the cholesterol depletion from the cell membrane of human lung cancer cells A549 cells after the treatment with saturated CDE1 solution; Natural ligand E₁ (40 μM); Positive control methyl-β-cyclodextrin (Methyl-CD) and β-cyclodextrin (beta-CD); Doxorubicin (DOX) at 0.5, 2.5 μM; Adamantane (AD) at 10, 100 μM; and the drug complex CDE1-Ada-DOX in different host/guest ratio of 1:1, 1:3 and 1:5 respectively, at the host concentration of 10 μM. Cell cultures were treated for 2 h at 37° C. to deplete cholesterol. Cells were then washed twice in serum-free media and lysed. Cholesterol levels were detected using EnzyChrom™ cholesterol assay kit.

FIG. 21 is a graph of the cholesterol depletion from the cell membrane of human normal lung cells T-80 cells after the treatment with saturated CDE1 solution; Natural ligand E₁ (40 μM); Positive control methyl-β-cyclodextrin (Methyl-CD) and β-cyclodextrin (beta-CD); Doxorubicin (DOX) at 0.5, 2.5 μM; Adamantane (AD) at 10, 100 μM; and the drug complex CDE1-Ada-DOX in different host/guest ratio of 1:1, 1:3 and 1:5 respectively, at the host concentration of 10 μM. Cell cultures were treated for 2 h at 37° C. to deplete cholesterol. Cells were then washed twice in serum-free media and lysed. Cholesterol levels were detected using EnzyChrom™ cholesterol assay kit.

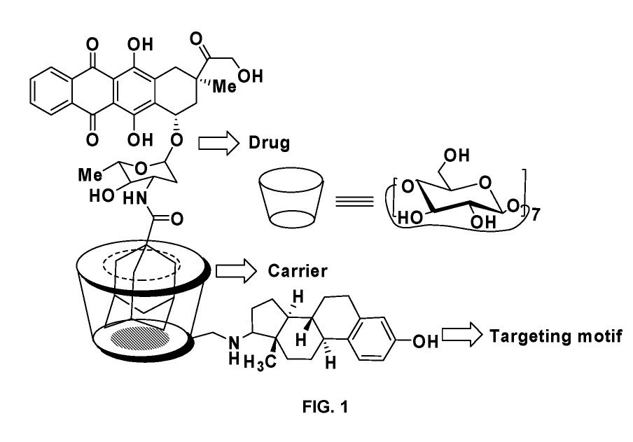

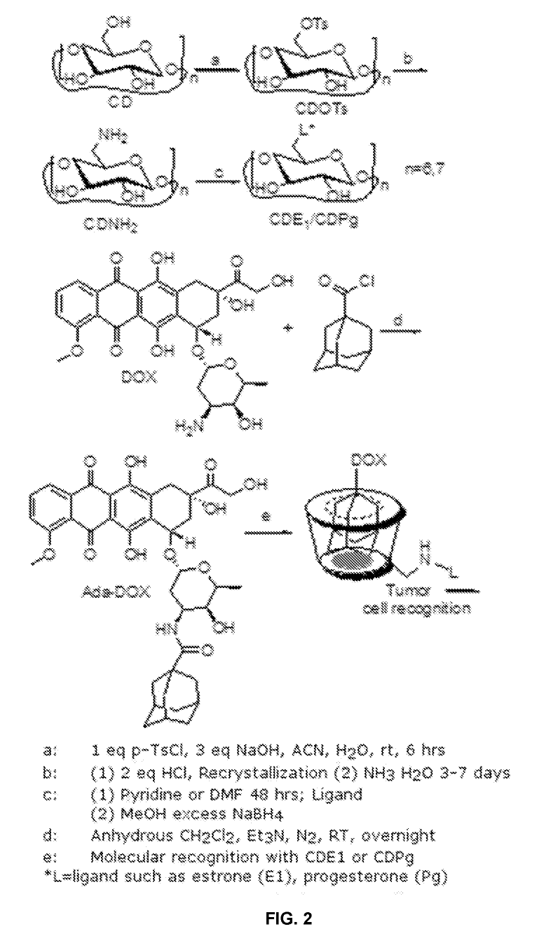

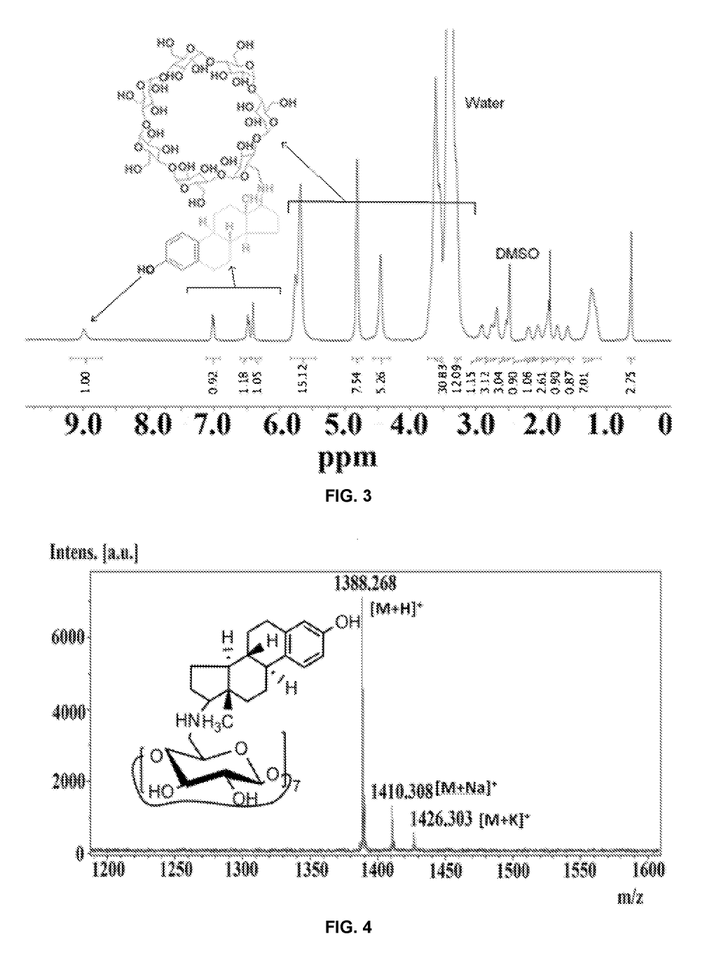

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting. It is also understood that, although the disclosure describes various embodiments to demonstrate specific aspects of the disclosure, the various embodiments may be combined without departing from the spirit of the disclosure, shall be within the scope of the present disclosure, and be protected by the accompanying claims.

### Definitions

The terms “bioactive agent” and “active agent”, as used interchangeably herein, include, without limitation, physiologically or pharmacologically active substances that act locally or systemically in the body. A bioactive agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they have been placed in a predetermined physiological environment.

The term “small molecule”, as used herein, generally refers to an organic molecule that is less than about 2000 g/mol in molecular weight, less than about 1500 g/mol, less than about 1000 g/mol, less than about 800 g/mol, or less than about 500 g/mol. Small molecules are non-polymeric and/or non-oligomeric.

The term “targeting moiety”, as used herein, refers to a moiety that binds to or localizes to a specific locale. The moiety may be, for example, a protein, nucleic acid, nucleic acid analog, carbohydrate, or small molecule. The locale may be a tissue, a particular cell type, or a subcellular compartment. The targeting moiety or a sufficient plurality of targeting moieties may be used to direct the localization of a particle or an active entity. The active entity may be useful for therapeutic, prophylactic, or diagnostic purposes.

The term “derivative” refers to any compound having the same or a similar core structure to the compound but having at least one structural difference, including substituting, deleting, and/or adding one or more atoms or functional groups. The term “derivative” does not mean that the derivative is synthesized from the parent compound either as a starting material or intermediate, although this may be the case. The term “derivative” can include salts, prodrugs, or metabolites of the parent compound. Derivatives include compounds in which free amino groups in the parent compound have been derivatized to form amine hydrochlorides, p-toluene sulfoamides, benzoxycarboamides, t-butyloxycarboamides, thiourethane-type derivatives, trifluoroacetylamides, chloroacetylamides, or formamides. Derivatives include compounds in which carboxyl groups in the parent compound have been derivatized to form salts, methyl and ethyl esters or other types of esters or hydrazides. Derivatives include compounds in which hydroxyl groups in the parent compound have been derivatized to form O-acyl or O-alkyl derivatives. Derivatives include compounds in which a hydrogen bond donating group in the parent compound is replaced with another hydrogen bond donating group such as OH, NH, or SH. Derivatives include replacing a hydrogen bond acceptor group in the parent compound with another hydrogen bond acceptor group such as esters, ethers, ketones, carbonates, tertiary amines, imine, thiones, sulfones, tertiary amides, and sulfides.

The term “reactive coupling group”, as used herein, refers to any chemical functional group capable of reacting with a second functional group to form a covalent bond. The selection of reactive coupling groups is within the ability of the skilled artisan. Examples of reactive coupling groups can include primary amines (—NH₂) and amine-reactive linking groups such as isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters. Most of these conjugate to amines by either acylation or alkylation. Examples of reactive coupling groups can include aldehydes (—COH) and aldehyde reactive linking groups such as hydrazides, alkoxyamines, and primary amines. Examples of reactive coupling groups can include thiol groups (—SH) and sulfhydryl reactive groups such as maleimides, haloacetyls, and pyridyl disulfides. Examples of reactive coupling groups can include photoreactive coupling groups such as aryl azides or diazirines. The coupling reaction may include the use of a catalyst, heat, pH buffers, light, or a combination thereof.

The terms “biocompatible” and “biologically compatible”, as used interchangeably herein, refer to materials that are, with any metabolites or degradation products thereof, generally non-toxic to the recipient, and cause no significant adverse effects to the recipient. Generally speaking, biocompatible materials are materials which do not elicit a significant inflammatory or immune response when administered to a patient. In some embodiments a biocompatible material elicits no detectable change in one or more biomarkers indicative of an immune response. In some embodiments, a biocompatible material elicits no greater than a 10% change, no greater than a 20% change, or no greater than a 40% change in one or more biomarkers indicative of an immune response.

The term “subject” refers to any individual who is the target of administration. The subject can be a vertebrate, for example, a mammal. Thus, the subject can be a human. The term does not denote a particular age or sex. The term “patient” refers to a subject afflicted with a disease or disorder. The term “patient” includes human and veterinary subjects.

The term “treating”, as used herein, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

The term “preventing”, as used herein includes both totally eliminating or partially reducing the risk of a disease, disorder or condition occurring in an animal which may be predisposed to the disease, disorder and/or condition but has not yet been diagnosed as having it. Preventing can include delaying the onset of a disease, disorder, or condition in an animal which may be predisposed to the disease, disorder and/or condition but has not yet been diagnosed as having it.

The terms “sufficient” and “effective”, as used interchangeably herein, refer to an amount (e.g. mass, volume, dosage, concentration, and/or time period) needed to achieve one or more desired result(s). For example, a therapeutically effective amount refers to an amount needed to achieve one or more therapeutic effects.

The term “pharmaceutically acceptable”, as used herein, refers to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration. A “pharmaceutically acceptable carrier”, as used herein, refers to all components of a pharmaceutical formulation which facilitate the delivery of the composition in vivo. Pharmaceutically acceptable carriers include, but are not limited to, diluents, preservatives, binders, lubricants, disintegrators, swelling agents, fillers, stabilizers, and combinations thereof.

“Parenteral administration”, as used herein, means administration by any method other than through the digestive tract or non-invasive topical or regional routes. For example, parenteral administration may include administration to a patient intravenously, intradermally, intraperitoneally, intrapleurally, intratracheally, intramuscularly, subcutaneously, subjunctivally, by injection, and by infusion.

“Topical administration”, as used herein, means the non-invasive administration to the skin, orifices, or mucosa. Topical administrations can be administered locally, i.e. they are capable of providing a local effect in the region of application without systemic exposure. Topical formulations can provide systemic effect via adsorption into the blood stream of the individual. Topical administration can include, but is not limited to, cutaneous and transdermal administration, buccal administration, intranasal administration, intravaginal administration, intravesical administration, ophthalmic administration, and rectal administration.

### Transdermal

“Enteral administration”, as used herein, means administration via absorption through the gastrointestinal tract. Enteral administration can include oral and sublingual administration, gastric administration, or rectal administration.

“Pulmonary administration”, as used herein, means administration into the lungs by inhalation or endotracheal administration. As used herein, the term “inhalation” refers to intake of air to the alveoli. The intake of air can occur through the mouth or nose.

Inclusion Complexes and Formulations Thereof

Inclusion complexes are provided containing a targeted carrier having an inclusion host that is non-covalently associated with an active agent or precursor thereof. The targeted carrier can include a conjugate of an inclusion host and a targeting moiety. In various embodiments the conjugate can include a polymer, e.g. wherein the targeting moiety is conjugated to the polymer, to the host, or both. The targeting moiety can be covalently bound to the inclusion host. The targeting moiety can be covalently bound to a polymer that is covalently bound to the inclusion host. The inclusion complexes can form by self-assembly of the targeted carrier and the active agent or precursor thereof. The active agent can bind non-covalently to the inclusion host or can contain a binding partner that can bind non-covalently to the inclusion host.

In exemplary embodiments, the targeted complex includes an inclusion complex having a cyclodextrin inclusion host, having an estrogen or progesterone targeting moiety attached thereto. In various embodiments, the active agent is doxorubicin, wherein a precursor of the active agent can include adamantane covalently attached to the doxorubicin. In some embodiments, the targeted carrier includes a polymer covalently associated with the cyclodextrin.

Inclusion Hosts

The inclusion complex can contain an inclusion host. The inclusion host can be capable of forming a strong non-covalent interaction with a binding partner. In some embodiments the inclusion host binds non-covalently with the binding partner with an association constant of about 500 M⁻¹ or greater, about 1,000 M⁻¹ or greater, about 1,200 M⁻¹ or greater, or about 1,500 M⁻¹ or greater. For example, the association constant for the inclusion host-binding partner binding can be about 500-5,000 M⁻¹, about 1,000-5,000 M⁻¹, or about 1,200-3,000 M⁻¹. The inclusion host can bind the binding partner with dissociation constants of about 0.005 M or less, about 0.001 M or less, about 0.0005 M or less, or about 0.0001 M or less.

The inclusion host can be a cyclodextrin. The terms “cyclodextrin” and “cycloamylose” are used interchangeably herein to refer to cyclic oligosaccharides having from 5-20, 5-10, or 6-8 α-D-glucopyranoside units linked 1->4. The cyclodextrin can be an α-cyclodextrin (6-membered ring), a β-cyclodextrin (7-membered ring), or a γ-cyclodextrin (8-membered ring), the structures of which are shown below.

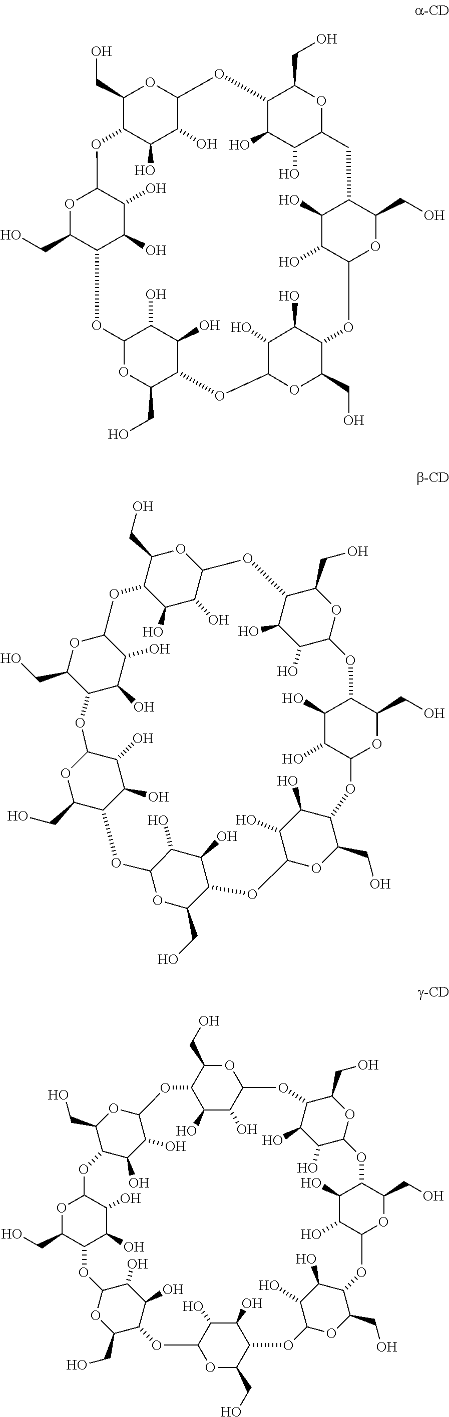

The inclusion host can be a crown ether. The term “crown ether”, as used herein, refers broadly to cyclic ethers and common derivatives thereof. Common derivative include those where the oxygen atoms have been partially or completely substituted with nitrogen, sulfur, or phosphorous.

The inclusion host can be a calixarene. The term “calixarene” is used herein to refer generally to macrocycles of phenol groups ortho-linked by short alkylene bridges such as methylene or ethylene. Calixarenes will typically have 3-10, 4-10, or 4-8 phenol groups in the ring.

The inclusion host can be a cucurbituril. The term “cucurbituril”, as used herein, refers to a macrocyle composed of glycoluril monomer linked by a pair of methylene bridges. The cucurbituril can have a structure

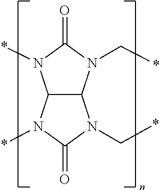

where n is an integer, typically 4-10, 4-8, or 5-7.

Targeting Moieties

The targeted carrier includes one or more targeting moieties. The targeting moiety can be conjugated to the inclusion host or can be conjugated to a polymer or linker that is conjugated to the inclusion host.

The targeting moiety can be a peptide. The targeting moiety can be a protein such as an antibody or an antigen-binding fragment thereof. The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody. The targeting moiety of the conjugate can be an antibody or antigen binding fragment thereof. The targeting elements moieties should have an affinity for a cell-surface receptor or cell-surface antigen on the target cells. The targeting moiety can be a nucleic acid targeting moiety. The targeting moiety can be a small molecule. The targeting moieties may result in internalization of the particle within the target cell.

The targeting moiety can specifically recognize and bind to a target molecule specific for a cell type, a tissue type, or an organ. The target molecule can be a cell surface polypeptide, lipid, or glycolipid. The target molecule can be a receptor that is selectively expressed on a specific cell surface, a tissue or an organ. Cell specific markers can be for specific types of cells including, but not limited to stem cells, skin cells, blood cells, immune cells, muscle cells, nerve cells, cancer cells, virally infected cells, and organ specific cells. The cell markers can be specific for endothelial, ectodermal, or mesenchymal cells. Representative cell specific markers include, but are not limited to cancer specific markers.

The targeting moiety may specifically bind to an antigen that is expressed by tumor cells. The antigen expressed by the tumor may be specific to the tumor, or may be expressed at a higher level on the tumor cells as compared to non-tumor cells. Antigenic markers such as serologically defined markers known as tumor associated antigens, which are either uniquely expressed by cancer cells or are present at markedly higher levels (e.g., elevated in a statistically significant manner) in subjects having a malignant condition relative to appropriate controls, are contemplated for use in certain embodiments.

Tumor-associated antigens may include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erb B gene). Other tumor-associated antigens include molecules that may be directly involved in transformation events, or molecules that may not be directly involved in oncogenic transformation events but are expressed by tumor cells (e.g., carcinoembryonic antigen, CA-125, melonoma associated antigens, etc.).

A tumor antigen can include a cell surface molecule. Tumor antigens of known structure and having a known or described function, include the following cell surface receptors: HER1 (GenBank Accession No. U48722), HER2 (Yoshino, et al., J. Immunol., 152:2393 (1994); Disis, et al., Canc. Res., 54:16 (1994); GenBank Acc. Nos. X03363 and M17730), HER3 (GenBank Acc. Nos. U29339 and M34309), HER4 (Plowman, et al., Nature, 366:473 (1993); GenBank Acc. Nos. L07868 and T64105), epidermal growth factor receptor (EGFR) (GenBank Acc. Nos. U48722, and KO3193), vascular endothelial cell growth factor (GenBank No. M32977), vascular endothelial cell growth factor receptor (GenBank Acc. Nos. AF022375, 1680143, U48801 and X62568), insulin-like growth factor-I (GenBank Acc. Nos. X00173, X56774, X56773, X06043, European Patent No. GB 2241703), insulin-like growth factor-II (GenBank Acc. Nos. X03562, X00910, M17863 and M17862), transferrin receptor (Trowbridge and Omary, Proc. Nat. Acad. USA, 78:3039 (1981); GenBank Acc. Nos. X01060 and M11507), estrogen receptor (GenBank Acc. Nos. M38651, X03635, X99101, U47678 and M12674), progesterone receptor (GenBank Acc. Nos. X51730, X69068 and M15716), follicle stimulating hormone receptor (FSH-R) (GenBank Acc. Nos. Z34260 and M65085), retinoic acid receptor (GenBank Acc. Nos. L12060, M60909, X77664, X57280, X07282 and X06538), MUC-1 (Barnes, et al., Proc. Nat. Acad. Sci. USA, 86:7159 (1989); GenBank Acc. Nos. M65132 and M64928) NY-ESO-1 (GenBank Acc. Nos. AJ003149 and U87459), NA 17-A (PCT Publication No. WO 96/40039), Melan-A/MART-1 (Kawakami, et al., Proc. Nat. Acad. Sci. USA, 91:3515 (1994); GenBank Acc. Nos. U06654 and U06452), tyrosinase (Topalian, et al., Proc. Nat. Acad. Sci. USA, 91:9461 (1994); GenBank Acc. No. M26729; Weber, et al., J. Clin. Invest, 102:1258 (1998)), Gp-100 (Kawakami, et al., Proc. Nat. Acad. Sci. USA, 91:3515 (1994); GenBank Acc. No. S73003, Adema, et al., J. Biol. Chem., 269:20126 (1994)), MAGE (van den Bruggen, et al., Science, 254:1643 (1991)); GenBank Acc. Nos. U93163, AF064589, U66083, D32077, D32076, D32075, U10694, U10693, U10691, U10690, U10689, U10688, U10687, U10686, U10685, L18877, U10340, U10339, L18920, U03735 and M77481), BAGE (GenBank Acc. No. U19180; U.S. Pat. Nos. 5,683,886 and 5,571,711), GAGE (GenBank Acc. Nos. AF055475, AF055474, AF055473, U19147, U19146, U19145, U19144, U19143 and U19142), any of the CTA class of receptors including in particular HOM-MEL-40 antigen encoded by the SSX2 gene (GenBank Acc. Nos. X86175, U90842, U90841 and X86174), carcinoembryonic antigen (CEA, Gold and Freedman, J. Exp. Med., 121:439 (1985); GenBank Acc. Nos. M59710, M59255 and M29540), and PyLT (GenBank Acc. Nos. J02289 and J02038); p97 (melanotransferrin) (Brown, et al., J. Immunol., 127:539-46 (1981); Rose, et al., Proc. Natl. Acad. Sci. USA, 83:1261-61 (1986)).

Additional tumor associated antigens include prostate surface antigen (PSA) (U.S. Pat. Nos. 6,677,157; 6,673,545); β-human chorionic gonadotropin β-HCG) (McManus, et al., Cancer Res., 36:3476-81 (1976); Yoshimura, et al., Cancer, 73:2745-52 (1994); Yamaguchi, et al., Br. J. Cancer, 60:382-84 (1989): Alfthan, et al., Cancer Res., 52:4628-33 (1992)); glycosyltransferase β-1,4-N-acetylgalactosaminyltransferases (GaINAc) (Hoon, et al., Int. J. Cancer, 43:857-62 (1989); Ando, et al., Int. J. Cancer, 40:12-17 (1987); Tsuchida, et al., J. Natl. Cancer, 78:45-54 (1987); Tsuchida, et al., J. Natl. Cancer, 78:55-60 (1987)); NUC18 (Lehmann, et al., Proc. Natl. Acad. Sci. USA, 86:9891-95 (1989); Lehmann, et al., Cancer Res., 47:841-45 (1987)); melanoma antigen gp75 (Vijayasardahi, et al., J. Exp. Med., 171:1375-80 (1990); GenBank Accession No. X51455); human cytokeratin 8; high molecular weight melanoma antigen (Natali, et al., Cancer, 59:55-63 (1987); keratin 19 (Datta, et al., J. Clin. Oncol., 12:475-82 (1994)).

Targeting moieties can include derivatives of known targeting moieties, for example derivatives having reactive coupling groups that can be used to bond the targeting moiety to the inclusion host or to a polymer or other molecule. In some embodiments the targeting moiety is estrogen, progesterone, or a derivative thereof. In some embodiments the targeting moiety targets the estrogen receptor, the progesterone receptor, or both.

Polymers

The targeted carrier can include one or more polymers conjugated to the inclusion host, conjugated to the targeting moiety, or both. Suitable polymers can include polyalkanoic acids such as poly(lactic acid), poly (glycolic acid), or copolymers and derivatives thereof. Suitable polymers can include poly(alkylene oxides) such as poly(ethylene oxide), poly(propylene oxide), and copolymers and derivatives thereof. The polymers can include copolymers of a polyalkanoic acids and a poly(alkylene oxides) such as PEG-PLGA.

Active Agent

The conjugates contain one or more active agents, e.g. one or more therapeutic, prophylactic, or diagnostic agents. The active agent can be covalently bonded to a binding partner capable of forming a strong non-covalent binding with the inclusion host. In some embodiments the active agent can non-covalently bind the inclusion host without the binding partner. The association of the active agent with the inclusion host is preferably labile such that the inclusion complex can release the active agent at the appropriate time and location to be effective. For example, the association can be such that a therapeutically effective amount of a therapeutic agent is released at the site to achieve one or more beneficial therapeutic effects. In some embodiments the non-covalent interaction with the inclusion host is such that the active agent and, optionally, the binding partner are released at the appropriate time and location. In some embodiments the covalent bond between the binding partner and the active agent is labile and releases the active agent at the appropriate time and location. The active agent can be an anthracycline such as doxorubicin.

The active agent can be a therapeutic agent. Exemplary therapeutic agents include, but are not limited to, small molecules, organometallic compounds, nucleic acids, proteins (including multimeric proteins, protein complexes, etc.), peptides, lipids, carbohydrates, hormones, metals, radioactive elements and compounds, drugs, vaccines, immunological agents, etc., and/or combinations thereof. In some embodiments, the therapeutic agent is a small molecule and/or organic compound with pharmaceutical activity.

In some embodiments, the therapeutic agent is a clinically-used drug. In some embodiments, the drug is an anti-cancer agent, antibiotic, anti-viral agent, anti-HIV agent, anti-parasite agent, anti-protozoal agent, anesthetic, anticoagulant, inhibitor of an enzyme, steroidal agent, steroidal or nonsteroidal anti-inflammatory agent, antihistamine, immunosuppressant agent, anti-neoplastic agent, antigen, vaccine, antibody, decongestant, sedative, opioid, analgesic, anti-pyretic, birth control agent, hormone, prostaglandin, progestational agent, anti-glaucoma agent, ophthalmic agent, anti-cholinergic, analgesic, anti-depressant, anti-psychotic, neurotoxin, hypnotic, tranquilizer, anti-convulsant, muscle relaxant, anti-Parkinson agent, antispasmodic, muscle contractant, channel blocker, miotic agent, anti-secretory agent, anti-thrombotic agent, anticoagulant, anti-cholinergic, β-adrenergic blocking agent, diuretic, cardiovascular active agent, vasoactive agent, vasodilating agent, anti-hypertensive agent, angiogenic agent, modulators of cell-extracellular matrix interactions (e.g. cell growth inhibitors and anti-adhesion molecules), inhibitor of DNA, RNA, or protein synthesis, etc. Exemplary therapeutic agents that can be incorporated into the particles include, but are not limited to, tumor antigens, CD4+ T-cell epitopes, cytokines, chemotherapeutic agents, radionuclides, small molecule signal transduction inhibitors, photothermal antennas, monoclonal antibodies, immunologic danger signaling molecules, other immunotherapeutics, enzymes, antibiotics, antivirals (especially protease inhibitors alone or in combination with nucleosides for treatment of HIV or Hepatitis B or C), anti-parasitics (helminths, protozoans), growth factors, growth inhibitors, hormones, hormone antagonists, antibodies and bioactive fragments thereof (including humanized, single chain, and chimeric antibodies), antigen and vaccine formulations (including adjuvants), peptide drugs, anti-inflammatories, immunomodulators (including ligands that bind to Toll-Like Receptors to activate the innate immune system, molecules that mobilize and optimize the adaptive immune system, molecules that activate or up-regulate the action of cytotoxic T lymphocytes, natural killer cells and helper T-cells, and molecules that deactivate or down-regulate suppressor or regulatory T-cells), agents that promote uptake of the particles into cells (including dendritic cells and other antigen-presenting cells), nutraceuticals such as vitamins, and oligonucleotide drugs (including DNA, RNAs, antisense, aptamers, small interfering RNAs, ribozymes, external guide sequences for ribonuclease P, and triplex forming agents).

Representative anti-cancer agents include, but are not limited to, alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and decetaxel and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), topoisomerase inhibitors (including camptothecins such as camptothecin, irinotecan, and topotecan as well as derivatives of epipodophyllotoxins such as amsacrine, etoposide, etoposide phosphate, and teniposide), antibodies to vascular endothelial growth factor (VEGF) such as bevacizumab (AVASTIN®), other anti-VEGF compounds; thalidomide (THALOMID®) and derivatives thereof such as lenalidomide (REVLIMID®); endostatin; angiostatin; receptor tyrosine kinase (RTK) inhibitors such as sunitinib (SUTENT®); tyrosine kinase inhibitors such as sorafenib (Nexavar®), erlotinib (Tarceva®), pazopanib, axitinib, and lapatinib; transforming growth factor-a or transforming growth factor-β inhibitors, and antibodies to the epidermal growth factor receptor such as panitumumab (VECTIBIX®) and cetuximab (ERBITUX®).

Prophylactic agents that can be included in the conjugates include, but are not limited to, antibiotics, nutritional supplements, and vaccines. Vaccines may be isolated proteins or peptides, inactivated organisms and viruses, dead organisms and viruses, genetically altered organisms or viruses, and cell extracts. Prophylactic agents may be combined with interleukins, interferon, cytokines, and adjuvants such as cholera toxin, alum, Freund's adjuvant, etc. Prophylactic agents can include antigens of such bacterial organisms as Streptococccus pneumoniae, Haemophilus influenzae, Staphylococcus aureus, Streptococcus pyrogenes, Corynebacterium diphtheriae, Listeria monocytogenes, Bacillus anthracis, Clostridium tetany, Clostridium botulinum, Clostridium perfringens, Neisseria meningitidis, Neisseria gonorrhoeae, Streptococcus nutans, Pseudomonas aeruginosa, Salmonella typhi, Haemophiius parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, Camphylobacter jejuni, and the like; antigens of such viruses as smallpox, influenza A and B, respiratory syncytial virus, parainfluenza, measles, HIV, varicella-zoster, herpes simplex 1 and 2, cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, and the like; antigens of fungal, protozoan, and parasitic organisms such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondil, Trichomonas vaginalis, Schistosoma mansoni, and the like. These antigens may be in the form of whole killed organisms, peptides, proteins, glycoproteins, carbohydrates, or combinations thereof.

Binding Partners

The active agent can be covalently bound to a binding partner that binds the inclusion host. The binding partner can bind the inclusion host via of π-bonding, cation-π, Van der Walls, hydrogen-bonding interactions, or a combination thereof. The binding partner can be a phenyl or substituted phenyl group. The binding partner can be naphthol or a naphthol derivative. The binding partner can be a lipid such as cholesterol. The binding partner can be a cage-like cycloalkane such as adamantane, diamantine, or iceane.

Pharmaceutical Formulations

Pharmaceutical formulations are provided that contain an effective amount of inclusion complexes in a pharmaceutical carrier appropriate for administration to an individual in need thereof. The formulations can be administered parenterally (e.g., by injection or infusion), topically (e.g., to the eye), or via pulmonary administration.

Parenteral Formulations

The inclusion complexes can be formulated for parenteral delivery, such as injection or infusion, in the form of a solution or suspension. The formulation can be administered via any route, such as, the blood stream or directly to the organ or tissue to be treated.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the inclusion complexes can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s) or inclusion complexes.

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the inclusion complexes in the required amount in the appropriate solvent or dispersion medium with one or more of the excipients listed above, as required, followed by filtered sterilization. Generally, dispersions are prepared by incorporating the various sterilized inclusion complexes into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the inclusion complexes plus any additional desired ingredient from a previously sterile-filtered solution thereof. The powders can be prepared in such a manner that the particles are porous in nature, which can increase dissolution of the particles. Methods for making porous particles are well known in the art.

Pharmaceutical formulations for parenteral administration are preferably in the form of a sterile aqueous solution or suspension of particles formed from one or more inclusion complexes Acceptable solvents include, for example, water, Ringer's solution, phosphate buffered saline (PBS), and isotonic sodium chloride solution. The formulation may also be a sterile solution, suspension, or emulsion in a nontoxic, parenterally acceptable diluent or solvent such as 1,3-butanediol.

In some instances, the formulation is distributed or packaged in a liquid form. Alternatively, formulations for parenteral administration can be packed as a solid, obtained, for example by lyophilization of a suitable liquid formulation. The solid can be reconstituted with an appropriate carrier or diluent prior to administration.

Solutions, suspensions, or emulsions for parenteral administration may be buffered with an effective amount of buffer necessary to maintain a pH suitable for ocular administration. Suitable buffers are well known by those skilled in the art and some examples of useful buffers are acetate, borate, carbonate, citrate, and phosphate buffers.

Solutions, suspensions, or emulsions for parenteral administration may also contain one or more tonicity agents to adjust the isotonic range of the formulation. Suitable tonicity agents are well known in the art and some examples include glycerin, mannitol, sorbitol, sodium chloride, and other electrolytes.

Solutions, suspensions, or emulsions for parenteral administration may also contain one or more preservatives to prevent bacterial contamination of the ophthalmic preparations. Suitable preservatives are known in the art, and include polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions for parenteral administration may also contain one or more excipients known art, such as dispersing agents, wetting agents, and suspending agents.

Topical Formulations

The inclusion complexes can be formulated for topical administration. Suitable dosage forms for topical administration include creams, ointments, salves, sprays, gels, lotions, emulsions, liquids, and transdermal patches. The formulation may be formulated for transmucosal, transepithelial, transendothelial, or transdermal administration. The compositions contain one or more chemical penetration enhancers, membrane permeability agents, membrane transport agents, emollients, surfactants, stabilizers, and combination thereof.

In some embodiments, the inclusion complexes can be administered as a liquid formulation, such as a solution or suspension, a semi-solid formulation, such as a lotion or ointment, or a solid formulation. In some embodiments, the inclusion complexes are formulated as liquids, including solutions and suspensions, such as eye drops or as a semi-solid formulation, such as ointment or lotion for topical application to the skin, to the mucosa, such as the eye or vaginally or rectally.

The formulation may contain one or more excipients, such as emollients, surfactants, emulsifiers, penetration enhancers, and the like.

“Emollients” are an externally applied agent that softens or soothes skin and are generally known in the art and listed in compendia, such as the “Handbook of Pharmaceutical Excipients”, 4th Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

Suitable classes of penetration enhancers are known in the art and include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols). Examples of these classes are known in the art.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase can contain a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

The difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of the inclusion complexes in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C₁₂-C₁₅ alkyl benzoates, mineral oil, squalane, cyclomethicone, capric/caprylic triglycerides, and combinations thereof.

Foams can include an emulsion in combination with a gaseous propellant. The gaseous propellant can include hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

In certain embodiments, it may be desirable to provide continuous delivery of one or more inclusion complexes to a patient in need thereof. For topical applications, repeated application can be done or a patch can be used to provide continuous administration of the noscapine analogs over an extended period of time.

Enteral Formulations

The inclusion complexes can be prepared in enteral formulations, such as for oral administration. Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations are prepared using pharmaceutically acceptable carriers. As generally used herein “carrier” includes, but is not limited to, diluents, preservatives, binders, lubricants, disintegrators, swelling agents, fillers, stabilizers, and combinations thereof. Polymers used in the dosage form include hydrophobic or hydrophilic polymers and pH dependent or independent polymers. Preferred hydrophobic and hydrophilic polymers include, but are not limited to, hydroxypropyl methylcellulose, hydroxypropyl cellulose, hydroxyethyl cellulose, carboxy methylcellulose, polyethylene glycol, ethylcellulose, microcrystalline cellulose, polyvinyl pyrrolidone, polyvinyl alcohol, polyvinyl acetate, and ion exchange resins.

Carrier also includes all components of the coating composition which may include plasticizers, pigments, colorants, stabilizing agents, and glidants.

Formulations can be prepared using one or more pharmaceutically acceptable excipients, including diluents, preservatives, binders, lubricants, disintegrators, swelling agents, fillers, stabilizers, and combinations thereof.

Delayed release dosage formulations can be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on excipients, materials, equipment and process for preparing tablets and capsules and delayed release dosage forms of tablets, capsules, and granules. These references provide information on carriers, materials, equipment and process for preparing tablets and capsules and delayed release dosage forms of tablets, capsules, and granules.

The inclusion complexes may be coated, for example to delay release once the particles have passed through the acidic environment of the stomach. Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water soluble polymer, water insoluble polymers and/or pH dependent polymers, with or without water insoluble/water soluble non polymeric excipient, to produce the desired release profile. The coating is either performed on dosage form (matrix or simple) which includes, but not limited to, tablets (compressed with or without coated beads), capsules (with or without coated beads), beads, particle compositions, “ingredient as is” formulated as, but not limited to, suspension form or as a sprinkle dosage form.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers are used to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

Methods of Making Inclusion Complexes and Formulations Thereof

The inclusion complexes can be made by any method known to those skilled in the art. In preferred embodiments the inclusion complex is formed by providing a targeted carrier having an inclusion host and an active agent that self assembles through non-covalent interactions with the inclusion host. In some embodiments the active agent has a binding partner for the inclusion host covalently attached such that the binding partner forms a strong non-covalent bond with the inclusion host.

The targeted carrier can be prepared by providing an inclusion host that is “activated” by having a reactive coupling group. For example, the inclusion host can have a reactive amine group. A targeting moiety having a group that forms a covalent bond with the reactive coupling group can be provided to form the targeted carrier. The active agent can be functionalized with a binding partner that binds non-covalently with the inclusion host to form the inclusion complex.

Methods of Using Inclusion Complexes and Formulations Thereof

The inclusion complexes and formulations thereof can be administered to a subject or patient in need thereof. In particular, the inclusion complexes and formulations thereof can be administered to a patient in need of a therapeutic effect. In preferred embodiments the patient has cancer and the therapeutic effect is alleviating one or more symptoms associated with the disease, increasing the overall life expectancy of the patient, decreasing the tumor size, or a combination thereof.

The inclusion complexes can release the active agent at the target site. For example, the inclusion complexes can release an anti-cancer agent at the site of the tumor

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, nanotechnology, organic chemistry, biochemistry, botany and the like, which are within the skill of the art. Such techniques are explained fully in the literature.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Chemicals and Reagents

α and β-Cyclodextrin hydrate and cerim (IV) sulphate tetrahydrate were purchased from Acros Organics (Thermo Fisher Scientific, Waltham, Mass.). Ammonium molybdate (para) tetrahydrate was purchased from Alfa Aesar Inc. (Ward Hill, Mass.). p-Toluenesulfonyl chloride, estrone (E1), progesterone (Pg), doxorubicin hydrochloride, 4′,6-Diamidino-2-phenylindole dihydrochloride (DAPI), 1-adamantanecarbonyl chloride, ammonium bicarbonate, sodium hydroxide, ammonia hydroxide, ninhydrin, hydrochloric acid, iodine, sulfuric acid, acetic acid, chloroform-d, dimethyl sulfoxide-d₆, and CM sephadex C25 were all purchased from Sigma-Aldrich Chemicals Co. (St. Louis, Mo.). The Pro-Prep™ Protein extraction kit was purchased from iNtRon Biotechnology Inc. (Kyungki-Do, Korea). Spectra/Por dialysis membrane with a molecular weight cutoff of 3,000 Da was purchased from Spectrum Laboratories (Rancho Dominguez, Calif.). Unless indicated otherwise, all solvents for chromatographic isolation were of analytical grade. HPLC-grade acetone, acetonitrile (ACN), ethyl acetate (EtOAc), methanol, 1-propanol (1-PA), pyridine, dimethylformamide (DMF), dimethyl sulfoxide (DMSO) and thin layer chromatography plates (1,000 mm and 200 mm) were purchased from Fisher Scientific Co. (Fair lawn, NJ). Heat-inactivated fetal bovine serum, fetal bovine serum, and newborn calf serum were purchased from Hyclone Laboratories Inc. (Logan, Utah). Estrogen receptor alpha (D8H8) rabbit, p-P44/42 Mark (T202/Y204) (20G11) rabbit and β-actin antibody were purchased from Cell Signaling Technology, Inc. (Danvers, Mass.). HRP mouse anti-rabbit IgG monoclonal antibody was purchased from ProSci Inc. (San Diego, Calif.). Pierce ECL western blotting substrate was purchased from Thermo Fisher Scientific Inc. (Waltham, Mass.).

### Instruments:

Nuclear Magnetic Resonance (NMR): Unless otherwise indicated, all ¹H/¹³C NMR spectra were recorded on a high-resolution digital NMR Spectrospin Bruker Avance 500 MHz and 800 MHz (Bruker BioSpin Co., The Woodlands, Tex.).

Fourier Transform Infrared Spectroscopy (FTIR): In all cases, unless otherwise indicated, the instrument used for FTIR analysis was a Perkin-Elmer 1725 series FTIR spectrometer (Perkin-Elmer Co., Norwalk, Conn.) equipped with a room temperature deuterated triglycine sulfate detector and controlled by a Perkin-Elmer 7300 PC. The software used for collecting the FTIR data was the Spectrum version 5.3.1.

UV/vis spectra: In all cases, unless otherwise indicated, the UV/vis spectra were recorded using a Perkin-Elmer Lambda 2 UV/vis double beam spectrophotometer.

Release kinetics: In all cases, unless otherwise indicated, the release kinetics of the drugs as well as protein content detection were measured by the Synergy H4 hybrid multi-mode microplate reader coupled with Gen5 2.0 data analysis software, and Take 3 nano-drop plate (BioTek Instruments Inc. Winooski, Vt.).

Mass spectroscopy: In all cases, unless otherwise indicated, the mass spectra were obtained using a Bruker Daltronics Autoflex MALDI-TOF (Bruker Daltonics Inc., Billerica, Mass.), with α-acyano-4-hydroxycinnamic acid (α-CHCA) as the matrix (10.0 mg/ml, from Thermo Scientific Pierce). Data analysis was performed with the mMass program. ESI spectra were acquired by 6430 Triple Quad LC/MS system, and data analysis was performed with Agilent MassHunter workstation B.04.00.

Circular Dichroism Analysis: In all cases, unless otherwise indicated, the circular dichroism spectra were recorded at 25° C. in an Aviv model 215 circular dichroism spectrometer (Aviv Biomedicals Inc., Lakewood, N.J.) using a 0.1 cm cell for three scans with 0.1 nm bandwidth.

Flow cytometer: In all cases, unless otherwise indicated, the flow cytometric analysis was performed on a FACS (Becton Dickinson Immunocytometry Systems, San Jose, Calif.) by counting 10,000 events.

Western Blot: In all cases, unless otherwise indicated, the western blots were visualized by Bio-Rad digital imaging system and analyzed by Quantity One 1-D analyzing software.

Transmission Electron Microscopy (TEM): In all cases, unless otherwise indicated, the TEM images were observed on JEM 100CX system; (JEOL Ltd, Tokyo, Japan) with an accelerating voltage of 160 kV.

Scanning Electron Microscopy (SEM): In all cases, unless otherwise indicated, the SEM images were recorded on JEOL JSM6490 scanning electron microscope.

### Example 1: Synthesis of Activated Cyclodextrins

Activated α- and β-cyclodextrins were synthesized having reactive functional groups, including tosyl (—SO₂C₆H₄CH₃) groups and amine (—NH₂) groups.

Synthesis of mono-6-deoxy-6-(p-tolylsulfonyl)β-cyclodextrin (CDOTs): A solution of p-toluenesulfonyl chloride (846.3 mg, 4.44 mmol) in 5 ml ACN was added to 80 ml aqueous solution of β-CD (5.0 g, 4.44 mmol) and NaOH (434.8 mg, 10.8 mmol) dropwise over 15 min. After stirring for 4 hrs at ° C. in N₂ atmosphere, the solution was neutralized by adding 0.6 ml of 2.0 N aqueoushydrochloric acid and the product was recrystallized at 4° C. overnight, and then washed with acetone. CDO-Ts was obtained in a yield of 84.6%. The product were tracked by TLC (1-propanol:ethyl acetate:water:NH₃.H₂O=3:1:2:1 (v/v)) visualized with cerium-ammonium-molybdate (CAM) solution.

Synthesis of mono-6-deoxy-6-amino-β-cyclodextrin (CDNH₂): Ammonia solution (30 ml) was added into crude CDOTs (3 g) and the solution stirred for 3 days at 50° C. The final solution was recrystallized in cold acetone to give the crude compound CDNH₂ which was further purified by cation exchange column with deionized water and 0.1 M (NH₄)₂CO₃ aqueous buffer solution with overall yield 79%. The product were tracked by TLC (1-propanol:ethyl acetate:water:NH₃.H₂O=3:1:2:1 (v/v)) visualized with CAM solution.

Synthesis of mono-6-deoxy-6-(p-tolylsulfonyl)-α-cyclodextrin (α-CDOTs): p-Toluenesulfonyl chloride (634.0 mg, 2.96 mmol) was added to 50 ml pyridine solution of α-CD (5.0 g, mmol). After stirring overnight at room temperature in N₂ atmosphere, the solution was evaporated and recrystallized in cold acetone from water. Alpha-CDOTs was obtained in a yield of 73.4%. The product were tracked by TLC (1-propanol:ethyl acetate:water:NH₃.H₂O=3:1:2:1 (v/v)) visualized with CAM solution.

Synthesis of mono-6-deoxy-6-amino-α-cyclodextrin (α-CDNH₂): Ammonia solution (25 ml) was added into crude α-CDOTs (2 g) and the solution stirred for 3 days at 50° C. The final solution was recrystallized in cold acetone to give the crude compound α-CDNH₂ which was further purified by cation exchange column with gradient method, firstly, flushed with deionized water to remove unreacted α-CD, and then flushed with 0.1 M (NH₄)₂CO₃ aqueous buffer, pure α-CDNH₂ was obtained with yield 35.4%. The product were tracked by TLC (1-propanol:ethyl acetate:water:NH₃.H₂O=3:1:2:1 (v/v)) visualized with CAM.

The structure of all compounds was ambiguously determined by HRMS, ¹H- and ¹³C-NMR, as well as FTIR and UV-vis spectroscopy

### Example 2: Synthesis of Targeted Cyclodextrins

Targeted cyclodextrin polymers were prepared starting from the activated cyclodextrins prepared in Example 1. Targeting moieties included estrogen (E₁) and progesterone (Pg).

Chemical Synthesis of CDE₁: A mixture of β-CDNH₂ (200 mg, 0.177 mmol) and 4 equivalents of estrone (200 mg, 0.740 mmol) in anhydrous pyridine (or DMF) (3 ml) was flushed with N₂ and stirred vigorously at room temperature for 48 hrs. The mixture was evaporated and re-dissolved in methanol, excess NaBH₄ was then added, and the mixture was stirred overnight. At the end of the reaction, the product was extracted with water from ethyl acetate, following by cationic exchange flash column chromatography separation eluting with water, preparative TLC, and re-crystallization, finally afforded product CDE₁ (30.4 mg, 12.41%, Rf_{CDE1/CDNH2}=2.29). ¹H NMR (800 MHz, d₆-DMSO): 1.21, 1.86, 2.48, 3.28, 3.54, 3.59, 3.69, 4.45, 4.80, 5.66, 5.73, 6.40, 6.46, 7.01, 8.98 ppm; ¹³C NMR (100 MHz, d₆-DMSO) 11.61, 23.17, 26.25, 27.18, 29.09, 39.52, 51.73, 59.98, 72.45, 81.63, 102.09, 112.80, 114.99, 126.10, 130.58, 137.29, 154.91 ppm. UV-Vis λₘₐₓ/nm (H₂O): 295 nm; IR v/cm⁻¹: 705.01, 932.12, 1022.60, 1076.93, 1150.66, 1368.48, 3186.18, 3274.59; HRMS (MALDI-TOF, m/z): [M+H]⁺ calculated for C₆₀H₉₃NO₃₆, 1388.56; observed, 1388.268.

Chemical Synthesis of CDPg A mixture of β-CDNH₂ (200 mg, 0.177 mmol) and 4 equivalents of progesterone (200 mg, 0.740 mmol) in anhydrous pyridine (or DMF) (3 ml) was flushed with N₂ and stirred vigorously at room temperature for 48 hrs. The mixture was evaporated and re-dissolved in methanol, excess NaBH₄ was then added, and the mixture was stirred overnight. At the end of the reaction, the product was extracted with water from ethyl acetate, following by cationic exchange flash column chromatography separation eluting with water, preparative TLC, and re-crystallization, finally afforded product CDE₁ (30.4 mg, 12.41%, Rf_{CDPg/CDNH2}=2.27). HRMS (MALDI-TOF, m/z): [M+Na]⁺ calculated for C₆₃H₁₀₁NNaO₃₅, 1454.6; observed, 1454.7390.

Chemical Synthesis of α-CDE₁: A mixture of α-CDNH₂ (17.7 mg, 0.018 mmol) and excess estrone (45.5 mg, 0.17 mmol) in anhydrous pyridine (2 ml) was flushed with N₂ and stirred vigorously at room temperature for 48 hrs. The mixture was evaporated and re-dissolved in methanol, excess NaBH₄ was then added. At the end of the reaction after overnight, the product was extracted with water from ethyl acetate, following by preparative TLC, (2.4 mg, 10.8%, Rf_{α-CDE1/α-CDNH2}=2.21). ESI-MS (m/z): [M+H]⁺ calculated for C₅₄H₈₄NO₃₀, 1226.51; observed, 1226.21.

Chemical Synthesis of α-CDPg A mixture of α-CDNH₂ (25.8 mg, 0.027 mmol) and excess progesterone (39.2 mg, 0.124 mmol) in anhydrous pyridine (1.5 ml) was flushed with N₂ and stirred vigorously at room temperature for 48 hrs. The mixture was evaporated and re-dissolved in methanol, excess NaBH₄ was then added. At the end of the reaction after overnight, the product was extracted with water from ethyl acetate, following by preparative TLC, (0.8 mg, 2.3%, Rf_{α-CDPg/α-CDNH2}=1.44). ESI-MS (m/z): [M.H₂O+H]⁺ calculated for C₅₇H₉₄NO₃₁, 1288.58; observed, 1288.5.

The structure of all compounds was ambiguously determined by HRMS, ¹H- and ¹³C-NMR, as well as FTIR and UV-vis spectroscopy

### Example 3: Drug Inclusion in Targeted Cyclodextrins

A conjugate of adamantane (Ada) and doxorubicin (DOX) was prepared. Ada-COCl was dissolved in anhydrate DCM mixed with Et₃N, and then stirred at room temperature for 3 hrs under N₂. Doxorubicin hydrochloride was then added, stirred overnight and separated by flash chromatography (CH₂Cl₂:CH₃OH=100:1).

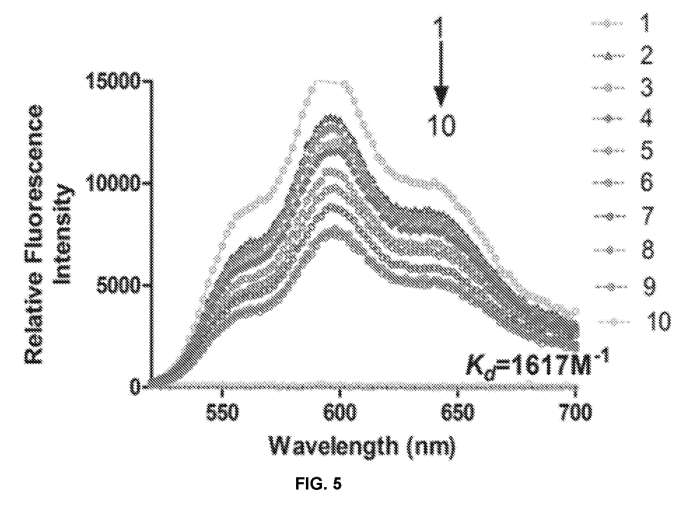

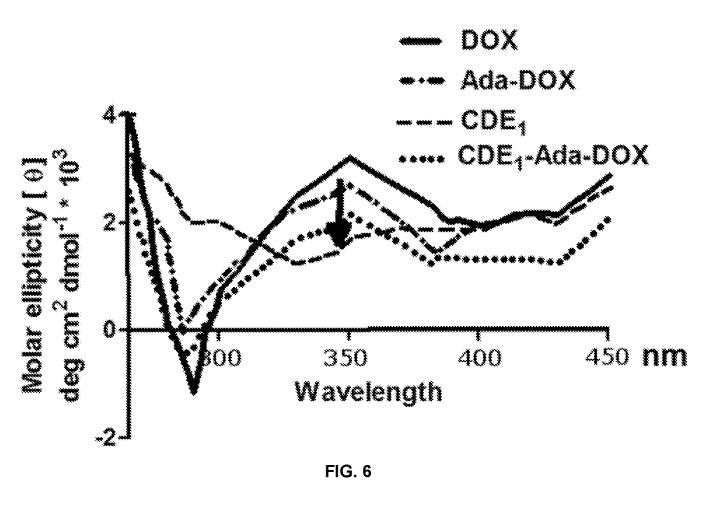

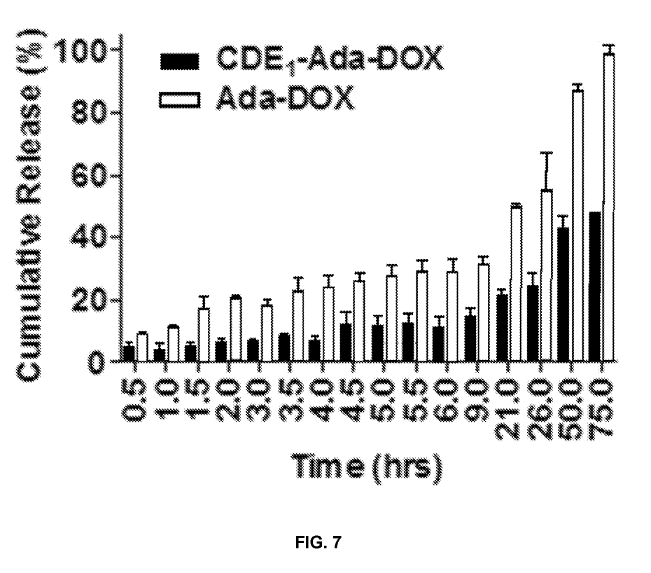

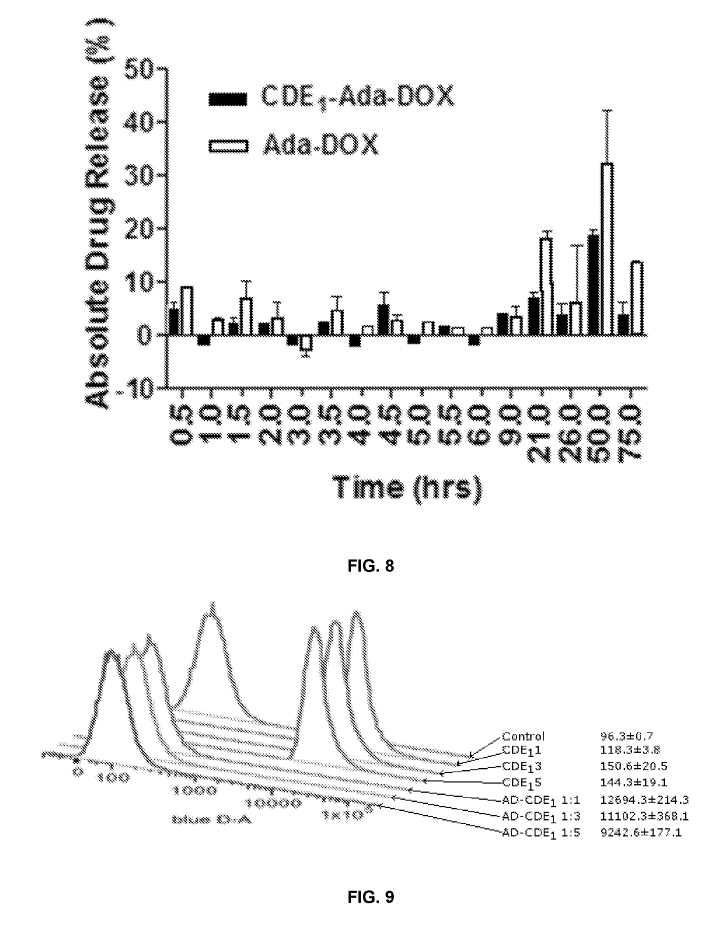

The inclusion complexes CDE₁-Ada-DOX, CDPg-Ada-DOX, α-CDE₁-Ada-DOX, and α-CDPg-Ada-DOX were readily formed using the Ada-DOX prodrug and the targeted cyclodextrins from Example 2. The 1:1 stoichiometric inclusion complex of CDE1 and Ada-DOX was easily formed by co-precipitation method and a strong association constant of CDE₁ and Ada-DOX was measured by fluorescence titration and found to be 1,617 M⁻¹ by the Scatchard plotting method (FIG. 5), demonstrating the strong affinity between adamantine and CDE₁. The strong binding ability was further confirmed by circular dichroism analyses (FIG. 6). These results demonstrate that the interactions between Ada-Dox with CDE₁ could change the conformation of the cyclodextrin cavity binding site and then alter the chiral microenvironment for the whole complex supramolecule. The release kinetics of Ada-Dox and CDE₁-Ada-Dox from the formulations clearly demonstrated that Ada-Dox release from CDE1-Ada-Dox was significantly slower than Ada-Dox within the incubation time period, where the amount of released drugs was quantified using a validated fluorescence method. In addition to the indication of the formation of the inclusion complex CDE1-Ada-DOX, the data revealed that CDE1-Ada-DOX possesses a continuously and sustained releasing feature (up to 46.12% for CDE1-Ada-DOX vs 99.00% for Ada-DOX after 75 h) compared to the parent prodrug of Ada-DOX (FIGS. 7 and 8).

### Example 4: Cellular Uptake of CDE₁-Ada-DOX in MCF-7 Cells

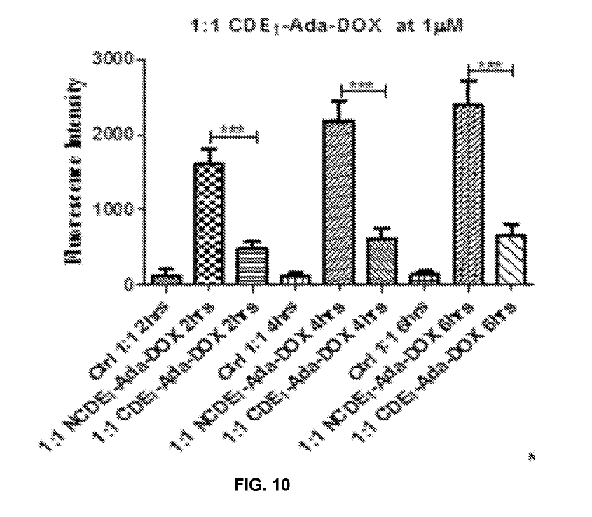

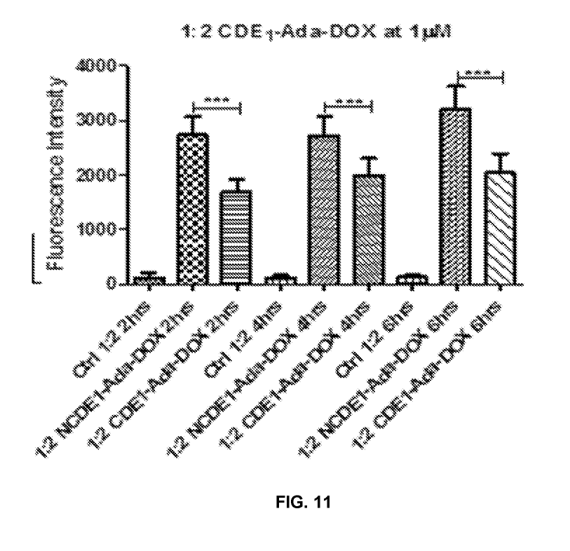

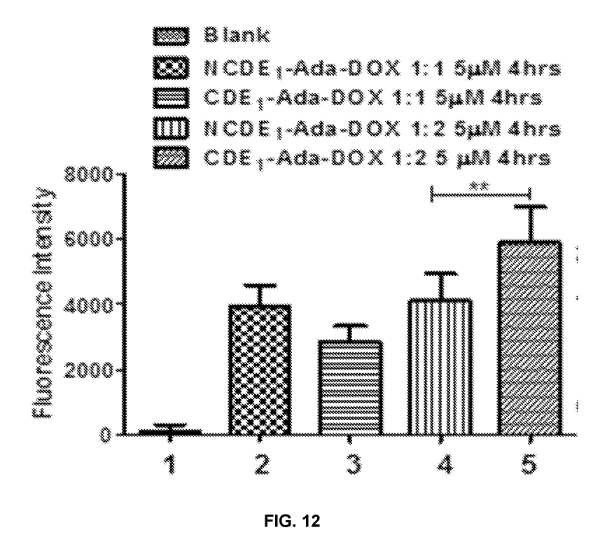

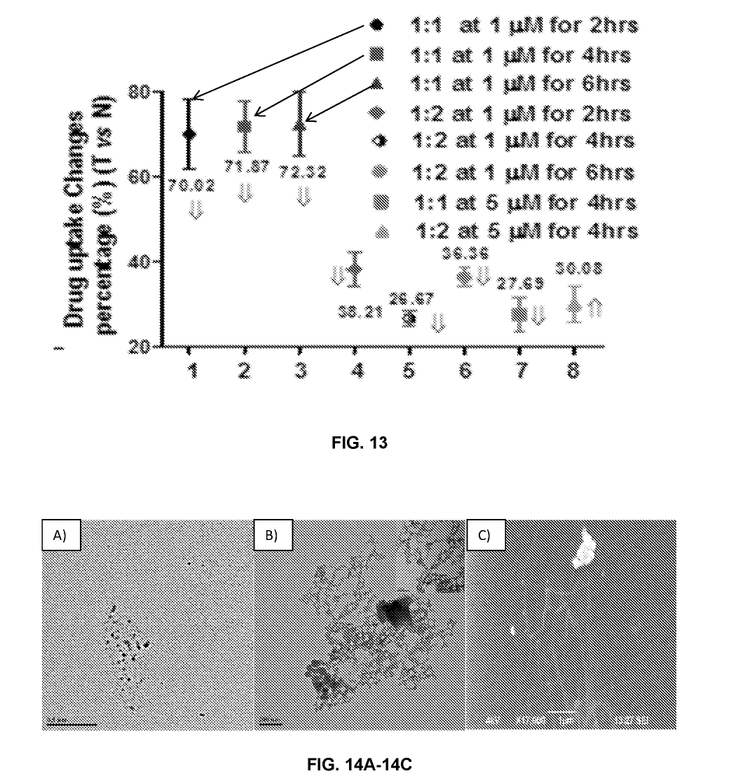

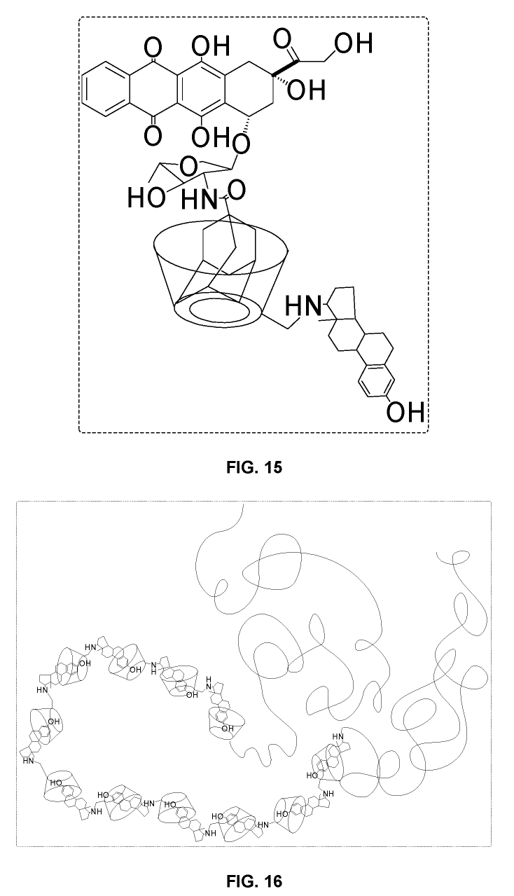

To investigate the cellular uptake of the ER targeting drug CDE₁-Ada-DOX, flow cytometry was performed using the ER-positive MCF-7 cells which take advantage of the intrinsic fluorescence of Ada-DOX. Cells incubated with DMEM medium only were used as the control. FIGS. 9-13 show representative quantitative flow cytometry results of the cellular uptake of the ER targeting drug CDE1-Ada-DOX and NCDE1-Ada-DOX which is the parent β-cyclodextrin and Ada-DOX inclusion without estrogen moiety attached. To elaborate the optimized formulation of the drug complex, MCF-7 cells were treated with CDE₁-Ada-DOX and NCDE₁-Ada-DOX at different host-guest ratios and drug concentrations for a certain period of time. The negative control cells without any drug treatment as well as the cyclodextrin vector CDE₁ showed only a low level of autofluorescence at all the time points. With the molar ratio over 1 between CDE₁ and Ada-DOX (FIG. 9), the drug complex uptakes by MCF-7 cells dropped considerably by 15% and 38% for 1:3 and 1:5 formulation compared to the 1:1 formulation respectively. Surprisingly, MCF-7 cells treated with targeting drug complex CDE₁-Ada-DOX showed lower uptakes than NCDE₁-Ada-DOX, which had no estrogen attached at 1 μM. The cells were exposed with drugs for 2, 4 and 6 hrs, respectively (FIG. 10). These unexpected and interesting findings led us to propose that estrogen residues that are covalently bonded with the cyclodextrin are stealthy under certain circumstances. When the host-guest ratio increase to 1:2, the internalization efficacy of the targeting drug CDE₁-Ada-DOX rebounded greatly compared with NCDE₁-Ada-DOX (FIG. 11). Furthermore, the drug uptakes of CDE₁-Ada-DOX exceeded that of NCDE₁-Ada-DOX when the concentration of the drug complex raised (FIG. 12). As the equilibrium of the supramolecular complex CDE₁-Ada-DOX favors products of CDE₁-Ada-Dox more than CDE₁, more targeting moieties are exposed to the mER which facilitates the drug internalization process. Altering equilibrium of the CDE₁ and drug inclusion, the anti-cancer drug was internalized in a controlled manner. The quantitative analysis of the drug uptake changes of CDE₁-Ada-DOX (T) over NCDE₁-Ada-DOX (N) are presented in FIG. 13. The targeting mER expressed on MCF-7 cells is clearly an effective means of affecting the uptake of the drug complex into these cells since the drug complex are taken up by receptor-mediated process. Overall, these results showed that conjugating estrogen to cyclodextrin preserved the binding ability after structure modification, and more importantly and notably, cellular uptake can be augmented significantly only if the estrogen moiety is adjusted to not be encapsulated inside the cyclodextrin as the competitive drugs are present. In other words, the estrogen residue is tailed outside the cyclodextrin cavity.

The intermolecular recognition between the covalent attached estrogen residue of one CDE₁ and the cyclodextrin of another CDE₁ molecule results in the host-guest ratio dependent difference in drug uptake since the targeting moiety has been entrapped by cyclodextrin and in turn lost its affinity to mERs. When an appropriate guest molecule, such as adamantine, approaches the cyclodextrin, the estrogen residues are pushed out and released. The competition between complexation of the drug and intermolecular inclusion of the estrogen in the cyclodextrin cavity leads to a concomitant drug uptake changes.

In order to further demonstrate the intermolecular recognition, transmission electron microscopes (TEM) and scanning electron microscopes (SEM) examinations were conducted. Representative TEM and SEM images of CDE1 and CDE1-Ada-DOX are shown in FIG. 14A-140.

In the absence of a guest molecule, the bonded estrogens reside inside the cyclodextrin cavity of the adjacent CDE1 molecule. The intermolecular assembly of CDE1 exhibits a tail-in-bucket structure and wire-like morphology. FIGS. 14B and 14C illustrate the long, tangled and uniform CDE1 wires. The amplified TEM image of the wire is shown in upper-left corner of FIG. 14B. The estrogen residue in CDE1 acts as linkers between the CDE1 molecules due to intermolecular recognition (FIG. 16). These processes continue to form the wire-like supramolecular structure. FIG. 14A shows a TEM image of a control experiment using CDE₁-Ada-DOX under the same preparation condition.

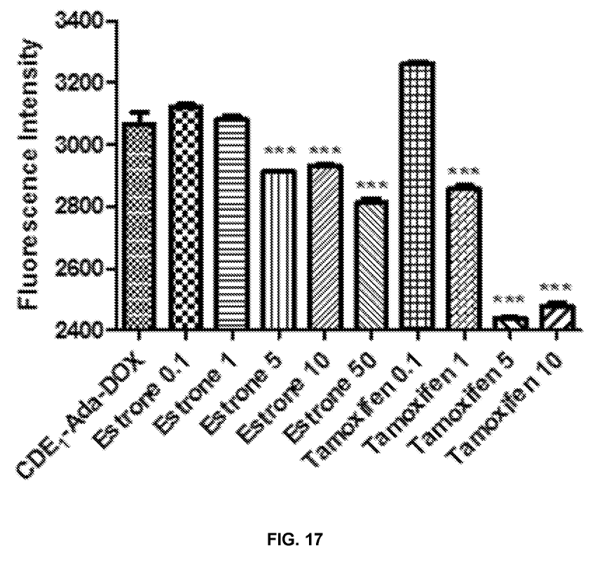

Moreover, the competition assay of estrone and tamoxifen with CDE₁-Ada-DOX in MCF-7 cells was conducted and is shown in FIG. 17. The flow cytometric analysis showed that estrone at 5-50 μM inhibited CDE1-Ada-DOX uptake in MCF-7 cells. Moreover, the ER antagonist-tamoxifen, which is the most prescribed medicine to treat hormone receptor-positive breast cancer, is capable of binding to the estrogen receptor but blocks its activity. Tamoxifen significantly inhibited CDE₁-Ada-DOX at 1 μM and above (p<0.001), 80% inhibition was observed at 5 μM. While for both estrone and tamoxifen, inhibition saturated at concentrations higher than 5 μM. These data suggest that CDE₁-Ada-DOX is internalized through an ER-mediated mechanism. Addition of an Ada-DOX guest led to the decomplexation of the estrogen moiety. In contrast, CDE₁-Ada-DOX particles showed an unorganized structure, since Ada-DOX competed with the estrogens of CDE₁-Ada-DOX, to release the ligands and disrupt the tail-in-bucket structure, resulting in an unorganized structure that we observed. The results are consistent and replicable with the TEM/SEM observations.

The mechanism of interaction of the estrogen-cyclodextrin conjugated with mER was further investigated in differentiating non-classical ligand activated mER actions versus the classical nER function. The agonist 17β-estradiol (E₂) is a key regulator of growth, differentiation, and biological function in reproductive tracts, mammary gland, and skeletal and cardiovascular systems via different ER pathways. In our study, E₂ was synthesized through reductive reaction from E1. E2 induces a number of rapid signaling events in cells devoid of classical ERs, but have mER present. Both treatments by E2 and CDE1-Ada-DOX resulted in rapid phosphorylation response of extracellular signal-regulated kinases 1 and 2 (ERK1 and ERK2). Cells treated with E₂ at 1 μM showed maximum MAPK phosphorylation in 10 min, while CDE1-Ada-DOX macromolecule treatment groups at 1 μM induced highest MAPK phosphorylationin 15 min, but declined thereafte. Therefore, these findings suggest CDE1-Ada-DOX interacts with mER and might also be used as a probe for studying non-genomic events mediated by mER, in addition to targeted drug delivery.

There is a potential to scavenge extracellular estrogen for the estrogen-cyclodextrin vectors, high affinity inclusion of extracellular estrogen to estrogen-cyclodextrin vectors after drug internalization is expected. It has been reported that cyclodextrin derivatives can extract cholesterol from lipid raft and elicit signal transduction, as well as serve to detect flavonoids, isoflavones

### Example 5: Cholesterol Depletion Studies

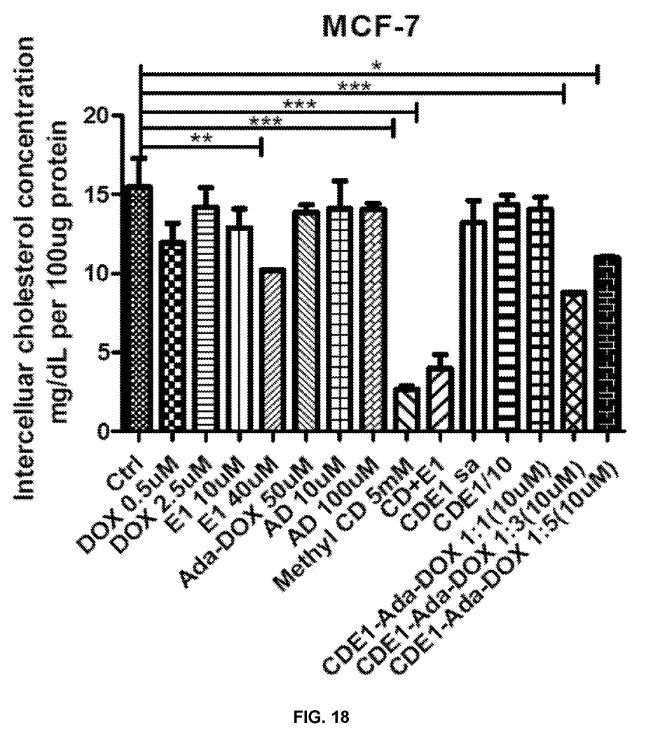

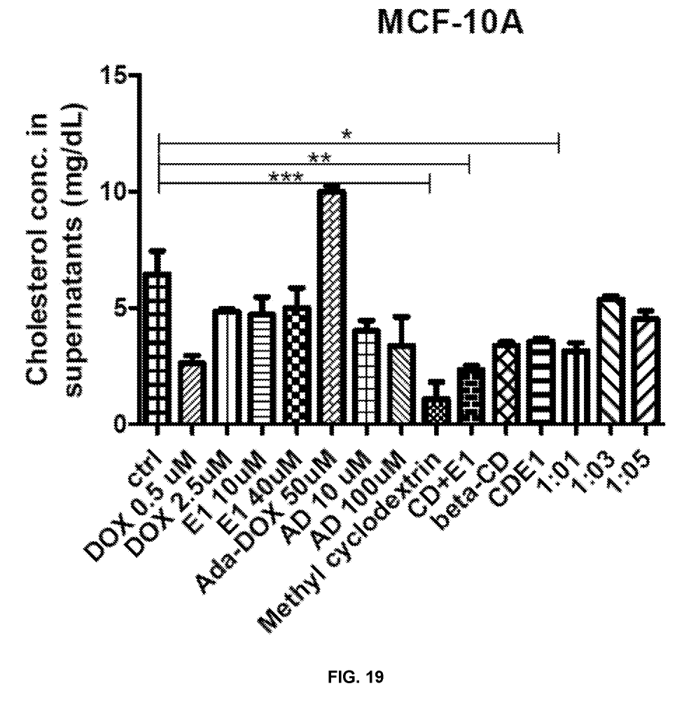

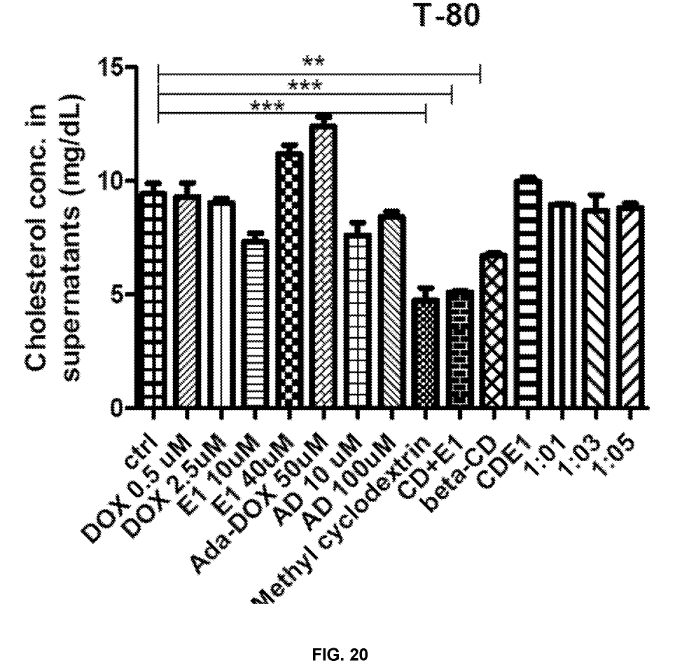

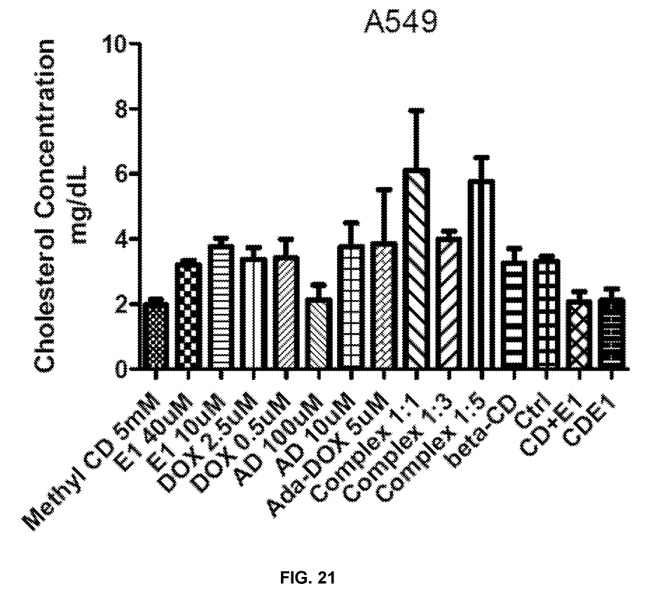

The cholesterol levels have been monitored in both cancerous cells (MCF-7 and A549 cells) and normal cells (MCF-10A and T80 cells) to investigate cholesterol depletion of lipid rafts on the cell membrane after drug exposure of CDE₁ and the drug complexes, since CD derivatives have been reported to be able to extract cholesterol from bilayer membrane by the CDs cavity, and modulates the activity of multiple signaling pathways. Cholesterol and estrogens have structural similarity. Cell cultures were treated for 2 h at 37° C. to deplete cholesterol. Cells were then washed twice in serum-free media and lysed. Cholesterol levels were detected using EnzyChrom™ cholesterol assay kit. The results are presented in FIGS. 18-21. It shows that the cholesterol level was not significantly affected after CDE₁ treatment due to the preoccupancy of the CD cavity by E₁ residues from the intramolecular self assembly.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

1. An inclusion complex comprising:
a targeted carrier comprising an inclusion host and having a targeting moiety attached thereto, and
an active agent or precursor thereof non-covalently associated with the inclusion host.

2. The inclusion complex of claim 1, wherein the inclusion host is selected from the group consisting of crown ethers, cyclodextrins, calixarenes, and cucurbituril.

3. The inclusion complex of claim 2, wherein the cyclodextrin is selected from the group consisting of α-cyclodextrin, β-cyclodextrin, γ-cyclodextrin, and combinations thereof.

4. The inclusion complex of claim 1, wherein the precursor comprises a conjugate of the active agent and a binding partner that non-covalently binds to the inclusion host.

5. The inclusion complex of claim 4, wherein the binding partner non-covalently binds to the inclusion host via an interaction selected from the group consisting of π-bonding, cation-π, Van der Walls, and hydrogen-bonding interactions.

6. The inclusion complex of claim 4, wherein the binding partner is selected from the group consisting of phenyl groups, lipids such as cholesterol, naphthol derivatives, and cage-like cycloalkanes such as adamantane, diamantine, and iceane.

7. The inclusion complex of claim 1, wherein the targeting moiety is selected from the group consisting of a protein, a peptide, a nucleic acid, and a small molecule.

8. The inclusion complex of claim 1, wherein the targeting moiety targets a cell surface receptor that results in internalization of the active agent.

9. The inclusion complex of claim 1, wherein the targeting moiety is estrogen or progesterone.

10. The inclusion complex of claim 1, wherein the targeted carrier comprises a polymer covalently associated with the inclusion host, optionally wherein the targeting moiety is covalently associated with the polymer.

11. The inclusion complex of claim 10, wherein the polymer comprises a biodegradable polymer such as a poly(alkynoic acid) or a copolymer or derivative thereof.

12. The inclusion complex of claim 10, wherein the polymer comprises a poly(alkylene oxide) such as poly(ethylene oxide), poly(propylene oxide), or a copolymer or derivative thereof.

13. The inclusion complex of claim 1, wherein the inclusion host is a cyclodextrin;
wherein the targeting moiety is estrogen or progesterone;
wherein the active agent is doxorubicin; and
wherein the precursor comprises adamantane covalently attached to the doxorubicin;
optionally wherein the targeted carrier comprises a polymer covalently associated with the cyclodextrin.

14. A pharmaceutical formulation for treating or preventing a disease or disorder in a subject in need thereof comprising an effective amount of an inclusion complex according to claim 1 and a pharmaceutically acceptable solvent.

15. The pharmaceutical formulation of claim 14, wherein the disease or disorder is cancer.

16. A method of treating or preventing a disease or disorder in a subject in need thereof, comprising administering an effective amount of an inclusion complex according to claim 1 to the subject.

17. A method of making an inclusion complex according to claim 1, comprising providing a targeted carrier comprising an inclusion host and having a targeting moiety attached thereto, and
providing an active agent or a precursor thereof, wherein the active agent or precursor non-covalently associates with the inclusion host to form the inclusion complex.

18. The method of claim 17, further comprising providing the inclusion host having a reactive coupling group attached thereto; and
reacting the targeting moiety to form a covalent bond with the reactive coupling group to form the targeted carrier.

19. The method of claim 18, wherein the reactive coupling group is an amine and the targeting moiety comprises an amine-reactive group.

20. The method of claim 17, further comprising conjugating a binding partner to the active agent to form the precursor, wherein the binding partner non-covalently binds to the inclusion host via an interaction selected from the group consisting of π-bonding, cation-π, Van der Walls, and hydrogen-bonding interactions.
